(19) United States
(12) Patent Application Publication
(10) Pub. No.: US 2021/0322986 A1
(43) Pub. Date: Oct. 21, 2021

(54) PCR SUBSTRATE, PCR CHIP, PCR SYSTEM AND LIQUID DROPLETS PULL-OUT METHOD

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Wenliang YAO, Beijing (CN); Peizhi CAI, Beijing (CN); Yingying ZHAO, Beijing (CN); Le GU, Beijing (CN); Nan ZHAO, Beijing (CN); Haochen CUI, Beijing (CN)

(21) Appl. No.: 17/273,173

(22) PCT Filed: Jul. 3, 2020
(86) PCT No.: PCT/CN2020/100229

§ 371 Date: Mar. 3, 2021

(30) Foreign Application Priority Data
Jul. 5, 2019 (CN) 201910604628.5

(51) Int. Cl. B01L 3/00; C12Q 1/686; B01L 7/00

(52) U.S. Cl. CPC B01L 3/502784; C12Q 1/686; B01L 2300/18; B01L 2400/0415; B01L 7/525

(57) ABSTRACT

The present disclosure provides a PCR substrate, a PCR chip, a PCR system, and a liquid droplets pull-out method. The PCR substrate includes a first base; a driving structure disposed on the first base and configured to drive liquid droplets to move; where the first base includes an injection region, a stretching region and an amplification region, and the driving structure is configured to enable liquid in the injection region to form liquid droplets in the stretching region and enable the liquid droplets to move in the amplification region according to a predetermined track.

### CROSS-REFERENCE TO RELATED APPLICATIONS

The present application claims priority to the Chinese Patent Application No. 201910604628.5, filed on Jul. 5, 2019, the content of which is incorporated herein in its entirety by reference.

### TECHNICAL FIELD

The present disclosure relates to the field of DNA sequencing technology, and in particular to a PCR substrate, a PCR chip, a PCR system and a liquid droplets pull-out method.

### BACKGROUND

PCR (Polymerase Chain Reaction) is a molecular biological technology for amplifying a specific DNA (Deoxyribonucleic Acid) fragment, and it is essentially relating to the replication of DNA fragments and realizing DNA amplification, so it is widely used in DNA detection.

### SUMMARY

The present disclosure provides a PCR substrate, a PCR chip, and a liquid droplets pull-out method.

The PCR substrate includes a first base, a driving structure disposed on the first base and configured to drive liquid droplets to move; and the first base includes an injection region, a stretching region and an amplification region, and the driving structure is configured to enable liquid in the injection region to form liquid droplets in the stretching region and enable the liquid droplets to move in the amplification region according to a predetermined track.

In some implementations, the driving structure includes a plurality of driving electrodes configured to generate an electric field to drive liquid droplets to move; and the plurality of driving electrodes includes a plurality of injection driving electrodes arranged in an array and disposed in the injection region; a plurality of stretching driving electrodes disposed in the stretching region, the plurality of stretching driving electrodes include a plurality of rows of stretching driving electrodes disposed along a first direction, the first direction is a direction from the injection region to the stretching region, and there is an interval between any two rows of stretching driving electrodes; at least one row of amplification driving electrodes disposed in the amplification region corresponding to the rows of stretching driving electrodes in the stretching region.

In some implementations, the driving structure further includes a plurality of gate lines and a plurality of data lines disposed on the first base; a part of intersection points of the gate lines and the data lines are a plurality of effective intersection points, the plurality of driving electrodes are disposed at positions of the, a plurality of effective intersection points correspondingly, a switching element is further disposed at a position of each of the plurality of effective intersection points, a first terminal and a second terminal of the switching element are respectively coupled to the data line and the driving electrode at the effective intersection point, and the gate line at the effective intersection point is coupled to a control electrode of the switching element.

In some implementations, the gate lines extend in the first direction, the data lines extend in a second direction, and the first direction intersects with the second direction.

In some implementations, the PCR substrate further includes a planarization insulating layer covering the gate lines, the data lines and the switching element, and the driving electrodes are disposed on a side of the planarization insulating layer distal to the first base and electrically coupled to second electrodes of corresponding switching elements through vias penetrating through the planarization insulating layer.

In some implementations, the PCR substrate further includes a hydrophobic layer disposed on the driving electrodes, and the hydrophobic layer on the driving electrodes changes in hydrophilic and hydrophobic properties when different voltages are applied to the driving electrodes.

In some implementations, in the amplification region, a first row of driving electrodes and a second row of driving electrodes are disposed corresponding to each of the driving electrodes in the stretching region, where the first row of driving electrodes and a row of driving electrodes in the stretching region corresponding to the first row of driving electrodes are disposed in a same row; each of the second row of the driving electrodes are disposed on a same side of the first row of driving electrodes corresponding thereto.

In some implementations, a part of the driving electrodes in each of the second row of driving electrodes are embedded with primer probes.

In some implementations, each of the second rows of driving electrodes is divided into a plurality of segments along the first direction, each segment includes three driving electrodes, and a primer probe is embedded in the driving electrode at the middle position among the three driving electrodes.

In some implementations, a shape of an orthographic projection of each amplification driving electrode in the amplification region on the first base and a shape of an orthographic projection of each stretching driving electrode in the stretching region on the first base are both square and have a same side length, and an extending direction of one set of opposite sides of the square is the first direction.

In some implementations, a shape of an orthogonal projection of each driving electrode, in the injection region, disposed in the same row as the first row of driving electrodes on the first base is a square, a shape of an orthogonal projection of each remaining driving electrode on the first base is rectangle, wherein an extending direction of one set of opposite sides of the square is the first direction, and an extending direction of short sides of the rectangle is the first direction.

The PCR chip includes a PCR substrate and a sealing substrate opposite to the driving structure, the sealing substrate includes a second base and a common electrode on a side of the second base proximal to the first base; edge areas of the PCR substrate and the sealing substrate opposite to each other are sealed by a sealing member, and orthographic projections of the driving electrodes on the first base are surrounded by an orthographic projection of the sealing member on the first base, the PCR chip further includes a sample inlet hole and a sample outlet hole communicated with a region corresponding to the injection region.

In some implementations, a first barrier is further disposed on the second base at a position corresponding to a position between adjacent driving electrodes among a column of driving electrodes in the stretching region, which are closest to the injection region, and the first barrier hermetically contacts the PCR substrate and the sealing substrate.

In some implementations, in the amplification region, a first row of driving electrodes and a second row of driving electrodes are disposed corresponding to each of the driving electrodes in the stretching region, where the first row of driving electrodes and a row of driving electrodes in the stretching region corresponding to the first row of driving electrodes are disposed in a same row, each of the driving electrodes of the second row is disposed on a same side of the first row of driving electrodes corresponding to the second row of driving electrodes, and the PCR chip further includes a plurality of rows of second barrier members disposed along the first direction, each row of the second barrier members corresponds to a second row of driving electrodes, the second row of driving electrodes is divided into a plurality of segments by the second harder members corresponding to the second row of driving electrodes, where a plurality of driving electrodes are disposed in each segment of driving electrodes, the second harrier members are disposed on a surface of the sealing substrate proximal to the PCR substrate, and the length direction of the second barrier members is a column direction.

In some implementations, the sample inlet hole and the sample outlet hole are disposed in the sealing substrate and penetrate through the sealing substrate.

In some implementations, the PCR chip further includes an oil inlet hole and an oil outlet hole communicating with a region corresponding to the amplification region.

In some implementations, the oil inlet hole and the oil outlet hole are disposed in the sealing substrate and penetrate through the sealing substrate.

The PCR system includes the PCR chip or the PCR substrate; a temperature control structure configured to control temperatures at different positions of the predetermined track; and a capture unit configured to capture an image of liquid droplets to analyze a number of specific bases.

The liquid droplets pull-out method using the PCR chip or the PCR substrate includes: injecting a sample into the injection region; stretching the sample from the injection region to the stretching region to form a strip sample; and cutting off the strip sample to form liquid droplets.

In some implementations, the driving structure further includes: a plurality of gate lines and a plurality of data lines disposed on the first base; a part of intersection points of the gate lines and the data lines are a plurality of effective intersection points, the plurality of driving electrodes are disposed at positions of the plurality of effective intersection points, a switching element is further disposed at a position of each of the plurality of effective intersection points, a first terminal and a second terminal of the switching element are respectively coupled to the data line and the driving electrode at the effective intersection point, and the gate line at the effective intersection point is coupled to a control electrode of the switching element; the injecting the sample into the injection region includes: providing a turn-on voltage to the gate lines corresponding to the driving electrodes in the injection region, providing an effective voltage to the data lines corresponding to the driving electrodes in the injection region, and providing an ineffective voltage to remaining data lines corresponding to the driving electrodes in the injection region; the stretching the sample from the injection region to the stretching region to form the strip sample includes: providing a turn-on voltage to the gate lines corresponding to the driving. electrodes in the stretching region, and sequentially providing an effective voltage to corresponding data lines in the stretching region along a direction from the injection region to the stretching region; and the cutting off the strip sample to form liquid droplets includes: providing a turn-on voltage to the gate lines corresponding to the driving electrodes in the stretching region, providing an ineffective voltage to adjacent data lines at the middle position in the stretching region, and sequentially providing an effective voltage pulse to the data lines at two sides of the adjacent data lines in the stretching region along the direction distal to the adjacent data lines.

### BRIEF DESCRIPTION OF THE DRAWINGS

FIG. 1 is a top view of a part of the structure of a PCR substrate according to an embodiment of the present disclosure;

FIG. 2 is a cross-sectional view of a part of the structure of a PCR substrate according to an embodiment of the present disclosure

FIG. 3 is a top perspective view of a part of the structure of a PCR chip according to an embodiment of the present disclosure

FIG. 4 is a top view of a sealing substrate of a PCR chip according to an embodiment of the present disclosure;

FIG. 5 is a cross-sectional view the sealing substrate shown in FIG. 4 along line AA;

FIG. 6 is a flowchart of a liquid droplets pull-out method according to an embodiment of the present disclosure;

FIGS. 7a-7c are diagrams illustrating states of a liquid droplet at different stages of a liquid droplet pull-out method according to an embodiment of the disclosure.

### DETAILED DESCRIPTION

In order that a person skilled in the art would better understand the technical solutions of the present disclosure, the following detailed description is given with references to the accompanying drawings and the specific embodiments,

A PCR chip (e.g., digital PCR chip) is a device used to control a large number of PCR reaction processes. An existing PCR chip is of a structure in which micro-pores are formed in a silicon substrate by etching, which involves complicated processes such as surface modification and the like, and the cost is relatively high. In addition, the process of dispersing a sample into liquid droplets requires manual or mechanical scraping, which is complicated and uneven in liquid droplets volume. As such, a PCR chip, which can be simply manufactured, and can realize simple liquid droplets growth operation and uniform liquid droplets volume, is needed.

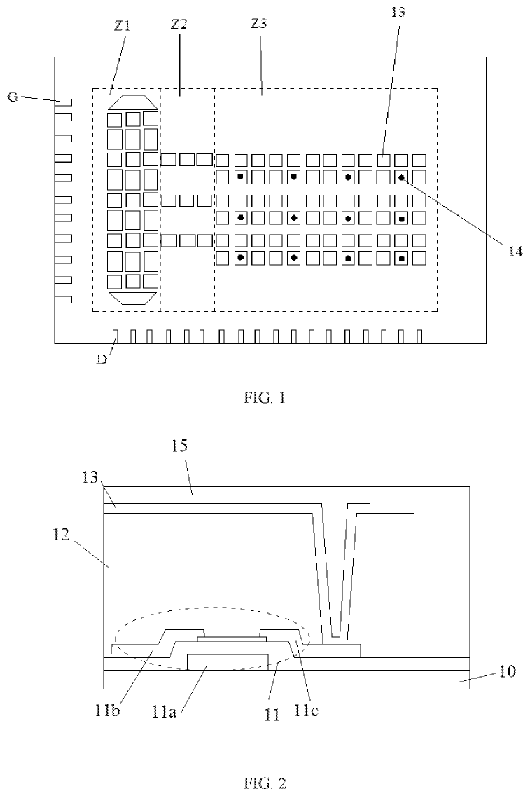

Referring to FIGS. 1 and 2, an embodiment of the present disclosure provides a PCR substrate (e.g., a PCR substrate) including: a first base 10; a driving structure disposed on the first base 10 for driving liquid droplets to move; the first base 10 includes an injection region Z1, a stretching region Z2 and an amplification region Z3, and the driving structure is configured to enable liquid in the injection region Z1 to form liquid droplets in the stretching region Z2 and enable the liquid droplets to move according to a predetermined track in the amplification region Z3.

When using the PCR substrate, a sample is firstly injected into the injection region Z1, then the sample is stretched in the stretching region Z2 to pull out liquid droplets, and then the liquid droplets moves according to a predetermined track in the amplification region Z3. Certainly, when the liquid droplets move in the amplification region Z3, it is necessary to set different temperatures at different positions of the amplification region Z3 to achieve DNA amplification.

For the control of the temperature, it may be implemented by a heating device (e.g. a resistance wire) and a detection device (e.g. a thermosensitive device) the PCR substrate, or may be implemented by a peripheral device other than the PCR substrate. The liquid droplets undergo a certain temperature cycle in the amplification region Z3 to realize DNA amplification. With the PCR substrate above, the control operation for the pull-out and movement of the liquid droplets is simpler, and since the PCR substrate can be manufactured by the existing semiconductor process, its functions are different and simpler.

Specifically, the driving structure includes a plurality of driving electrodes 13 configured to generate an electric field to drive the liquid droplets to move; and the injection region Z1 includes a plurality of driving electrodes 13 disposed in an array; the stretching region Z2 includes a plurality of rows of driving electrodes 13, each row of driving electrodes 13 includes a plurality of driving electrodes 13 disposed along a first direction, the first direction is a direction from the injection region Z1 to the stretching region Z2, and there is an interval between any two rows of driving electrodes 13; at least one row of driving electrodes 13 are disposed in the amplification region 13 corresponding to each row of driving electrodes 13 in the stretching region Z2.

When different voltages are applied to the driving electrodes 13, hydrophilic and hydrophobic properties of a hydrophobic layer 15 On the driving electrodes 13 are changed so as to guide the flow direction of the liquid droplets. For detailed driving timing can refer to the above-mentioned embodiment of the PCR system. Certainly, to achieve this, the voltage applied on each driving electrode 13 is required to be independently controlled.

In some implementations, the driving structure further includes: a plurality of gate lines and a plurality of data lines disposed on the first base 10; a part of intersection points of the gate lines and the data lines are effective intersection points, the driving electrodes 13 are disposed at positions of the effective intersection points accordingly, switching elements are further disposed at positions of the effective intersection points, two terminals of each switching element are respectively coupled to the data line and the driving electrode 13, and the gate line controls the connection and disconnection between the two terminals of the switching element.

As such, the gate line and the data line cooperate to realize the independent control of the driving voltage applied on each driving electrode 13.

In some implementations, the gate lines extend in a first direction, the data lines extend in a second direction, and the first direction intersects with the second direction.

Specifically, in a specific example provided by this embodiment, the first direction is a row direction, and the second direction is a column direction. In the following description, the switching element is a driving transistor 11 as an example.

A plurality of gate lines extending in the row direction and a plurality of data lines extending in the column direction are disposed on a first side of the first base 10, the gate lines and the data lines intersect each other in an insulating manner; at least part of the intersection points of the gate lines and the data lines are effective intersection points, each effective intersection point is provided with one driving transistor 11 and one driving electrode 13, a control electrode 11a of the driving transistor 11 is coupled to a corresponding gate line, a first electrode 11b of the driving transistor 11 is coupled to a corresponding data line, and a second electrode 11c of the driving transistor II is coupled to a corresponding driving electrode 13; the first base 10 is sequentially divided into an injection region Z1, a stretching region Z2 and an amplification region Z3 along the row direction; the intersection points in the injection region Z1 are all the effective intersection points; a part of rows of the intersection points in the stretching region Z2 are effective intersection points, and any different rows of the effective intersection points are spaced apart by at least one row of non-effective intersection points therebetween, that is, a plurality of rows of the effective intersection points spaced apart from each other in the stretching region Z2, and one row of non-effective intersection points is disposed between every two rows of effective intersection points; at least part of rows of the intersection points in the amplification region Z3 corresponding to the rows of the effective intersection points in the stretch region Z2 are first type effective intersection points.

In this case, the plurality of driving electrodes included in the driving structure includes: a plurality of injection driving electrodes arranged in an array and disposed in the injection region; a plurality of stretching driving electrodes disposed in the stretching region, the plurality of stretching driving electrodes include a plurality of rows of stretching driving electrodes disposed in the first direction, the first direction is a direction from the injection region to the stretching region, and an interval is arranged between every two rows of the stretching driving electrodes; and at least one row of amplification driving electrodes disposed in the amplification region corresponding to each row of stretching driving electrodes in the stretching region.

The row direction and the column direction in the present embodiment represent two directions intersect each other, and do not limit the two directions to be perpendicular. Each gate line controls the gate electrode 11a of the driving transistor 11 coupled thereto. Each data line is coupled to the first electrode 11b of the driving transistor 11 coupled thereto, The second electrode 11c of each driving transistor 11 is coupled to a driving electrode 13. Such connection relationship is similar to the connection relationship in a liquid crystal display substrate. A difference from the liquid crystal display substrate is that not all the intersection points of the gate lines and the data lines are provided with the driving transistors 11, that is, not all the intersection points of the gate lines and the data lines are effective intersections. The intersection points of the gale line and the data line where no driving transistor 11 is disposed are non-effective intersection points. According to the actual effect of each driving electrode 13 on liquid droplets operation in DNA sequencing, the arrangements of the driving electrodes 13 in the three regions of the injection region Z1, the stretching region Z2 and the amplification region Z3 are different, and the shapes of the driving electrode 13 may be the same or different according to actual requirements.

If the PCR substrate is applied to a PCR chip, the voltage applied on each driving electrode 13 can be independently controlled by independently controlling the signals on each gate line and each data line. The hydrophobic or hydrophilic property of the hydrophobic layer 15 on each driving electrode 13 can be controlled independently by cooperating with a common electrode 21. With this PCR substrate, the purpose of injecting a sample into the injection region Z1, pulling out liquid droplets in the stretching region Z2, and moving the liquid droplets in the amplification region Z3 can be achieved by changing the properties of the hydrophobic layer 15 on each driving electrode 13, the growth of liquid droplets is highly controllable, and the volumes of liquid droplets are uniform. The detailed liquid droplets pull-out method may refer to the following examples of the PCR system. The PCR substrate may be manufactured by adopting the manufacturing process and the manufacturing equipment of the existing liquid crystal display substrate or OLED display substrate, and the manufacturing process is simple. The hydrophobic layer 15 may be made of, for example, a dielectric layer (e.g., photoresist) and a fluoride.

Taking the current view of FIG. 1 as an example, after the PCR substrate is applied to the PCR chip, each driving electrode 13 or the temperature of regions where a plurality of driving electrodes 13 adjacent in the row direction are located may be independently controlled by an external device, so as to achieve DNA amplification.

Wires of the gate lines and the data lines are not shown in each drawing, and only the gate line bonding pads G and the data line bonding pads D are shown ire a frame region of the PCR substrate. Similar to the structure in the display substrate, a state of each gate line may be independently controlled by independently supplying a driving signal to each gate line bonding pad G. It is of course also possible to independently control a state of each data line by independently supplying a driving signal to each data line bonding pad D.

In an actual PCR substrate, the number of data lines in the amplification region Z3 may be up to several thousand, and FIG. 1 is only for showing the structure thereof.

In some implementations, the PCR substrate further includes a planarization insulating layer 12 covering the gate lines, the data lines, and the driving transistors 11. The driving electrodes 13 are disposed on a side of the planarization insulating layer 12 distal to the first base 10, and electrically coupled to a corresponding terminal of the corresponding switching element (e.g., the second electrode 11c of the driving transistor 11) through a via penetrating through the planarization insulating layer 12. The planarization insulating layer 12 plays a role of planarization on one hand, and on the other hand separates the data lines, the gate lines, the driving transistors 11 from the driving electrodes 13.

In some implementations, two rows of driving electrodes 13 are provided in the amplification region Z3 corresponding to each row of driving electrodes 13 in the stretching region Z2, where a first row of driving electrodes 13 in the amplification region Z3 face to the driving electrodes 13 in the stretching region Z2, and a second row of driving electrodes is located on a first side of the first row of driving electrodes (the side closer to the data line bonding pad D shown in FIG. 1). For example, the driving electrodes 13 of the stretching region Z2 and a part of the driving electrodes 13 in the amplification region Z3 are controlled by a same row of gate lines; while some gate lines do not correspond to any driving electrodes 13 in the stretching region Z2. but correspond to a row of driving electrodes 13 in the amplification region Z3 so as to control them. The above case is equivalent to the following case: in the amplification region Z3, at least part of the intersection points of the second row of driving electrodes that are adjacent to the first side of the first type effective intersection points are the second type effective intersection points. Taking FIG. 1 as an example, the amplification region Z3 in FIG. 1 includes six rows of driving electrodes 13. From top to bottom, the effective intersection points corresponding to the rows of driving electrodes 13 are the first type effective intersection points, the second type effective intersection points, the first type effective intersection points and the second type effective intersection points in sequence. When the liquid droplets move to the driving electrodes 13 corresponding to the second type effective intersection points, the cycle of PCR amplification can be completed; the liquid droplets only completes its movement on the driving electrode 13 corresponding to the first type effective intersection points. As such, the complexity of the supporting external system can be simplified. Certainly, the liquid droplets may also move only on the driving electrodes 13 corresponding to the first type effective intersection points and amplification can be completed simultaneously.

In some implementations, a part of the driving electrodes 13 in the second row of driving electrodes 13 are embedded with primer probes, which is equivalent to that the driving electrodes 13 corresponding to at least a part of the second type effective intersection points are embedded with primer probes 14. In the disclosed embodiment, the primer probes 14 may be disposed on the hydrophobic layer 15. When the liquid droplets move to the driving electrodes 13 corresponding to the second type effective intersection points embedded with the primer probe 14, the primer probes 14 can be dissolved into the liquid droplets, so that the catalytic reaction effect is realized. As such the PCR amplification operation is further simplified. For example, as shown in FIG. 1, the second row of driving electrodes is divided into a plurality of groups or segments along the first direction, each group or segment includes three driving electrodes, and the driving electrode at the middle position among the three driving electrodes is embedded with the primer probes.

In some implementations, a shape of an orthographic projection of each driving electrode 13 in the amplification region Z3 and in the stretching region Z2 on the first base 10 are square, and an extending direction of one set of opposite sides of each square is the first direction. In the current view of FIG. 1, the shapes of the driving electrodes 13 in the amplification region Z3 and the stretching region Z2 are square, and the extending direction of any side of the square is the row direction or the column direction. This arrangement is to improve the isotropy of the shapes of the liquid droplets in different directions parallel to the first base 10. The square driving electrode 13 has, for example, a side length of 50 μm.

In some implementations, in the injection region Z1, the shapes of the orthographic projections of the first row of driving electrodes 13 on the first base 10 are square, and the shapes of the orthographic projections of the second row of driving electrodes 13 on the first base 10 are rectangle, and the extending direction of the short side of the rectangle is the first direction, and the short side length of the rectangle may be set to be the same as the side length of the square. In the current view of FIG. 1, the driving electrodes 13 of the effective intersection points of the rows in the injection region Z1 corresponding to the rows of the first type effective intersection points in the stretching region Z2 are square, and the remaining driving electrodes 13 in the injection region Z1 are rectangular, and the extending direction of any side of the square is the row direction or the column direction, and the extending direction of the long side of the rectangle is the column direction. The size of the rectangular driving electrode 13 is for example 50×80 μm. Such arrangement is to facilitate the movement of the sample when the sample is injected. The spacing between the above driving electrodes 13 is, for example, 15 μm.

As shown in FIG. 1, the injection driving electrodes 13 in the injection region Z1 is distributed in injection region Z1 in an array, almost throughout the injection region Z1, in the stretching region Z2, the stretching driving electrodes 13 are disposed in a plurality of rows, and there is a space between every two adjacent rows of driving electrodes 13, for example, as shown in FIG. 1, for the first row of square injection driving electrodes 13 and the second row of rectangular injection driving electrodes 13 in the injection region Z1, only one row of square stretching driving electrodes 13 disposed in the same row as the first row of square injection driving electrodes 13 is disposed in the stretching region Z2, and the purpose of this arrangement is to make the circular liquid droplets injected in the injection region Z1 be stretched into narrower long liquid droplets in the stretching region so as to make them become small liquid droplets as shown in FIG. 7c, thereby achieving amplification in the amplification region Z3, as shown in the following FIGS. 7a to 7c. Therefore, in the amplification region, square amplification driving electrodes 13 are provided in the same row as the stretching driving electrodes 13 in the stretching region, and primer probes for amplifying a base sequence are provided at some parts in some of the amplification driving electrodes of the square amplification driving electrodes in the lower part thereof.

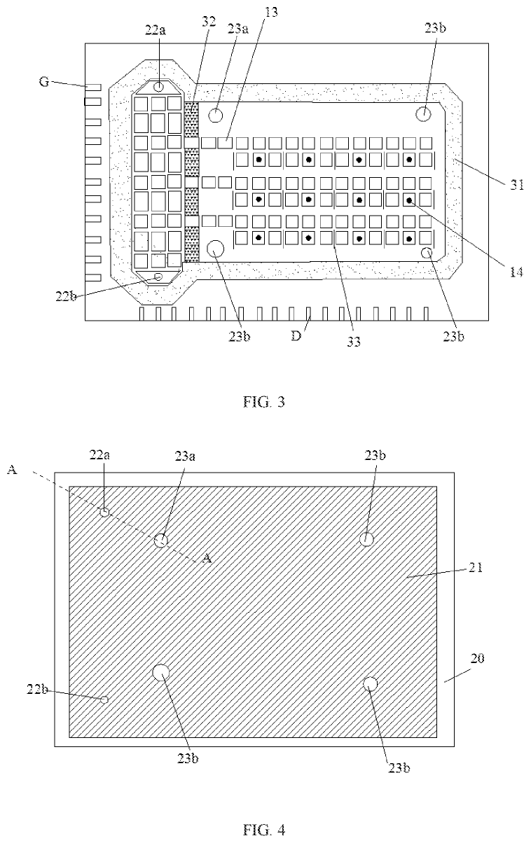

With the PCR substrate alone, the liquid droplets can be pulled out and the movement of the liquid droplets can be controlled, thereby amplifying the DNA. Certainly, a ground voltage relative to the voltage applied on the driving electrode 13 may be provided by a peripheral device or may be at infinity, However, as an embodiment, referring to FIGS. 3 to 5, the PCR substrate participates in constituting a PCR chip.

Referring to FIGS. 3 to 5 in combination with FIGS. 1 and 2, an embodiment of the present disclosure provides a PCR chip including: the PCR substrate in FIGS. 1 and 2 and a sealing substrate disposed proximal to the driving structure (for example, proximal to a side of the first base 10 disposed with the driving electrode), the sealing substrate includes a second base 20 and a common electrode 21 located on a side of the second base 20 proximal to the first base 10, and in practical applications, the voltage on the common electrode 21 may be grounded according to requirements; an edge areas of the PCR substrate and the sealing substrate opposite to each other are sealed by a sealing member 31, and an orthographic projection of the sealing member 31 on the first base 10 surrounds the orthographic projections of the driving electrodes 13 on the first base 10; the PCR chip further includes a sample inlet hole 22a and a sample outlet hole 22b communicating with a region corresponding to the injection region Z1.

The common electrode 21 is disposed opposite to the driving electrodes 13 and cooperates therewith to realize the control of the hydrophobic properties of the hydrophobic layer 15. The sealing member 31 defines a maximum space for the sample to move in the PCR chip. The sample can be injected into the injection region Z1 from the sample inlet hole 22a and discharged from the sample outlet hole 22b. The second base is made of acrylic transparent material, for example, and on which a layer of sodium polystyrene sulfonate is sprayed. The material of the common electrode 21 may be polyethylene dioxythiophene (PEDOT). A layer of dielectric material, such as resin, and a hydrophobic layer, such as Teflon, are spin coated on the common electrode 21. The height of the sealing member 31 is, for example, 30 μm.

The PCR chip has a simple structure, and the manufacturing process of the PCR chip is compatible with the manufacturing process of the existing display panel. In addition, easier operation on the liquid droplets can be achieved.

In some implementations, first barrier members 32 are further disposed between adjacent driving electrodes 13 in the column of driving electrodes 13, which is closest to the injection region Z1, in the stretching region Z2 and between two driving electrodes in the column of driving electrodes 13 closest to the sealing member 31 and the sealing member 31, the first barrier members 32 are in sealing contact with the PCR substrate and with the sealing substrate, and the first barrier members 32 may be disposed on corresponding positions of the second base 20 of the sealing substrate. That is, the first barrier members 32 define an opening that allows sample to enter from injection region Z1 into stretching region Z2. Parameters such as the shapes of first barrier members 32 and spacing between the first barrier members 32 may be set to be uniform to further facilitate to simultaneously pull out multiple liquid droplets with uniform volumes.

In some implementations, the PCR chip further includes a plurality of rows and columns of second barrier members 33 disposed on the sealing substrate, in the amplification region Z3, each row of second barrier members 33 corresponds to one second row of the driving electrodes 13, the second barrier members 33 divide the corresponding second row of the driving electrodes 13 into a plurality of segments, where each segment of driving electrodes 13 has a plurality of driving electrodes 13 therein, for example, each of the second row of driving electrodes is divided into a plurality of groups or segments along the first direction, each group or segment includes three driving electrodes, and the driving electrode at the middle position among the three driving electrodes is embedded with a primer probe. The second barrier members 33 are disposed on an outside surface of the PCR substrate proximal to the sealing substrate and a length direction thereof is the column direction, the length may be slightly larger than the side length of the driving electrodes 13, and may be, for example, 60 μm. In this embodiment, the liquid droplets are homogenized on the driving electrodes 13 corresponding to the second type effective intersection points, and the presence of the second barrier members 33 avoids cross-contamination during the homogenization process. If the height of the second barrier members 33 is up to reach the outside surface of the sealing substrate proximal to the PCR substrate, the second barrier members 33 simultaneously function to support a sealing cover plate.

In some implementations, the sample inlet hole 22a and the sample outlet hole 22b are provided in the sealing substrate and penetrate through the sealing substrate. That is, during use, the sample is injected and discharged from the sealing substrate. Certainly, the sample inlet hole 22a. and the sample outlet hole 22b may be provided in the sealing member 31.

In some implementations, the PCR chip further includes an oil inlet hole 23a and an oil outlet hole 23b communicating with a region corresponding to the amplification region Z3. Oily substance can be injected through the oil inlet hole 23a and discharged from the oil outlet hole 23b. The oily substance may fill the entire amplification region Z3 and stretching region Z2, thereby provides an external environment for the liquid droplets.

In some implementations, the oil inlet hole 23a and the oil outlet hole 23b penetrate through the sealing substrate. That is, the oily substance is injected and discharged from the sealing substrate during use. Certainly, the oil inlet hole 23a and the oil outlet hole 23b may be provided on the sealing member 31.

The present embodiment provides a PCR system, including: the PCR chip of FIGS. 3 to 5 or the PCR substrate of FIGS. 1 and 2; a temperature control structure configured to control the temperature at different positions on the preset track, for example, the temperature control structure may be a. semiconductor chilling plate disposed outside and below the first base of the PCR substrate; and an capture unit configured to capture an image of the liquid droplets to analyze the number of the specific bases, for example, the capture unit may be an external CCD or CMOS camera.

Certainly, the temperature control stricture may be integrated in the PCR substrate or the PCR chip, or may be a peripheral structure independent of the PCR chip or the PCR substrate. The operation control of the PCR system is simpler.

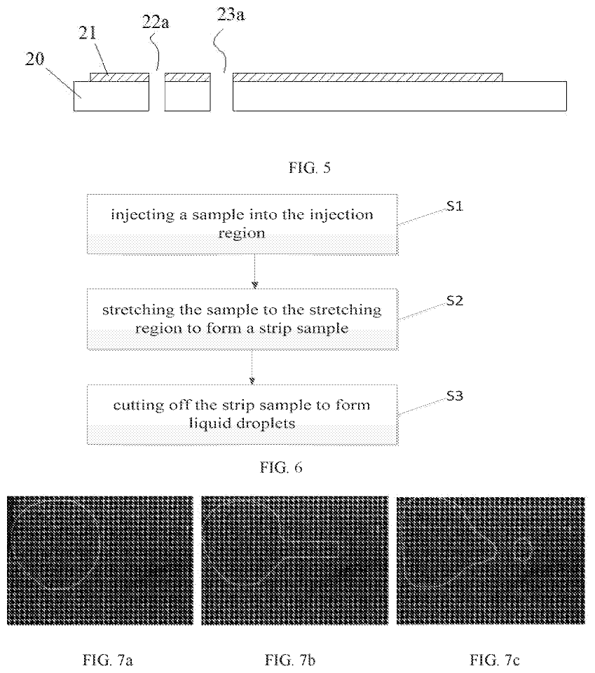

Referring to FIGS. 6 and 7a-7c, embodiments of the present disclosure provide a liquid droplets pull-out method. In FIGS. 7a-7c, the white lines indicate the profile of the sample and the liquid droplets. With the PCR chip of FIGS. 3 to 5, the liquid droplets pull-out method includes the following steps S1 to S3.

S1, injecting a sample into the injection region Z1. Specifically, referring to FIG. 7a, while the sample is injected from the sample inlet hole, a turn-on voltage is applied to each gate line, an effective voltage is applied to each data line corresponding to the injection region Z1, and an ineffective voltage is applied to the remaining data lines. As such, the hydrophobic layer 15 on the driving electrode 13 in the injection region Z1 exhibits hydrophilic property, thereby achieving injection of the sample. Certainly, the amount of sample injected can be controlled. The state shown in FIG. 7a is the state after the sample is injected into the injection region Z1 (actually, a large liquid droplet)

S2, stretching the sample to the stretching area Z2 to form a strip sample. Specifically, referring to FIG. 7b, the gate lines corresponding to the first type effective intersection points are supplied with a turn-on voltage, and the data lines corresponding to the stretching region Z2 are sequentially supplied with an effective voltage in a direction from the injection region Z1 toward the stretching region Z2. As shown in FIG. 7b, the hydrophobic layer 15 on the driving electrodes of the stretching region Z2 sequentially exhibits hydrophilic property, thereby guiding the sample to be stretched to be a strip sample.

S3, cutting off the strip sample to form liquid droplets. Specifically, referring to FIG. 7c, the gate lines corresponding to the first type effective intersection points are supplied with a turn-on voltage, adjacent data lines at a middle position in the stretching region Z2 are supplied with an ineffective voltage, and data lines at both sides of the adjacent data lines in the stretching region Z2 in a direction distal to the adjacent data lines are sequentially supplied with effective voltage pulses, such as the voltage pulses shown in FIG. 8. The strip sample is broken in the middle because the hydrophobic layer 15 on the driving electrodes at the positions of the middle area of the strip sample exhibits hydrophobic property again. As the property of the hydrophobic layer 15 on each driving electrode changes, the newly pulled-out liquid droplets completely separated from the sample in the injection region Z1. The effective voltage pulse applied here means that a square wave voltage signal is periodically applied to the driving electrode, so that the hydrophilic and hydrophobic properties of the hydrophobic layer on the driving electrode are alternately changed, thereby cutting off the strip sample and forming liquid droplets. The magnitude and frequency of the voltage pulse are generally determined according to the magnitude and number of the driving electrodes provided on the PCR substrate, and can be obtained by trial and error as required in actual applications.

The properties of the hydrophobic layer 15 on the driving electrodes 13 are controlled by the gate lines and the data lines so that the growth of the liquid droplets becomes controllable. Certainly, simultaneous growth of a plurality of liquid droplets could also be achieved. The entire PCR has a simple structure, and controllable liquid droplets generation, parallel growth, and high efficiency can be achieved.

It will be understood that the above embodiments are merely exemplary embodiments employed to illustrate the principles of the present disclosure, and the present disclosure is not limited thereto. It will be apparent to a person skilled in the art that various changes and modifications can be made therein without departing from the spirit and scope of the disclosure, and these changes and modifications are to be considered within the scope of the present disclosure.

1. A PCR substrate, comprising:
a first base; and
a driving structure disposed on the first base and configured to drive liquid droplets to move;
wherein the first base comprises an injection region, a stretching region and an amplification region, and the driving structure is configured to enable liquid in the injection region to form liquid droplets in the stretching region and enable the liquid droplets to move in the amplification region according to a predetermined track.

2. The PCR substrate of claim 1, wherein the driving structure comprises a plurality of driving electrodes configured to generate an electric field to drive liquid droplets to move; wherein
the plurality of driving electrodes comprises:
a plurality of injection driving electrodes arranged in an array and disposed in the injection region;
a plurality of stretching driving electrodes disposed in the stretching region, wherein the plurality of stretching driving electrodes comprise a plurality of rows of stretching driving electrodes disposed along a first direction, the first direction is a direction from the injection region to the stretching region, and there is an interval between any two rows of stretching driving electrodes; and
at least one row of amplification driving electrodes disposed in the amplification region corresponding to the rows of stretching driving electrodes in the stretching region.

3. The PCR substrate of claim 2, wherein
the driving structure further comprises a plurality of gate lines and a plurality of data lines disposed on the first base; a part of intersection points of the gate lines and the data lines are a plurality of effective intersection points, the plurality of driving electrodes are disposed at positions of the a plurality of effective intersection points correspondingly, a switching element is further disposed at a position of each of the plurality of effective intersection points, a first terminal and a second terminal of the switching element are respectively coupled to the data line and the driving electrode at the effective intersection point, and the gate line at the effective intersection point is coupled to a control electrode of the switching element.

4. The PCR substrate of claim 3, wherein the gate lines extend in the first direction, the data lines extend in a second direction, and the first direction intersects with the second direction.

5. The PCR substrate of claim 4, wherein the PCR substrate further comprises a planarization insulating layer covering the gate lines, the data lines and the switching element, and the driving electrodes are disposed on a side of the planarization insulating layer distal to the first base and electrically coupled to second electrodes of corresponding switching elements through vias penetrating through the planarization insulating layer.

6. The PCR substrate of claim 5, further comprising a hydrophobic layer disposed on the driving electrodes, wherein the hydrophobic layer on the driving electrodes changes in hydrophilic and hydrophobic properties when different voltages are applied to the driving electrodes.

7. The PCR substrate of claim 6, wherein in the amplification region, a first row of driving electrodes and a second row of driving electrodes are disposed corresponding to each row of the driving electrodes in the stretching region, wherein the first row of driving electrodes and a corresponding row of driving electrodes in the stretching region are disposed in a same row, the second row of the driving electrodes are disposed on a same side of the corresponding first row of driving electrodes.

8. The PCR substrate of claim 7, wherein a part of the driving electrodes in the second row of driving electrodes are embedded with primer probes.

9. The PCR substrate of claim 8, wherein the second row of driving electrodes is divided into a plurality of segments along the first direction, each segment comprises three driving electrodes, and a primer probe is embedded in the driving electrode at the middle position among the three driving electrodes.

10. The PCR substrate of claim 8, wherein a shape of an orthographic projection of each amplification driving electrode in the amplification region on the first base and a shape of an orthographic projection of each stretching driving electrode in the stretching region on the first base are both square and have a same side length, and an extending direction of one set of opposite sides of the square is the first direction.

11. The PCR substrate of claim 10, wherein a shape of an orthogonal projection of each driving electrode, in the injection region, disposed in the same row as the first row of driving electrodes on the first base is a square, a shape of an orthogonal projection of each remaining driving electrode on the first base is rectangle, wherein an extending direction of one set of opposite sides of the square is the first direction, and an extending direction of short sides of the rectangle is the first direction.

12. A PCR chip, comprising: the PCR substrate of claim 1 and a sealing substrate opposite to the driving structure, the sealing substrate comprising a second base and a common electrode on a side of the second base proximal to the first base; an edge area of the PCR substrate and the sealing substrate opposite to each other is sealed by a sealing member, and orthographic projections of the driving electrodes on the first base are surrounded by an orthographic projection of the sealing member on the first base, the PCR chip further comprises a sample inlet hole and a sample outlet hole communicating with a region corresponding to the injection region.

13. The PCR chip of claim 12, wherein a first barrier member is further disposed on the second base at a position corresponding to a position between adjacent driving electrodes among a column of driving electrodes, which are closest to the injection region, in the stretching region, and the first barrier hermetically contacts the PCR substrate and the sealing substrate.

14. The PCR chip of claim 12, wherein in the amplification region, a first row of driving electrodes and a second row of driving electrodes are disposed corresponding to each row of the driving electrodes in the stretching region, wherein the first row of driving electrodes and the corresponding row of driving electrodes in the stretching region are disposed in a same row, the second row of the driving electrodes are disposed on the same side of the corresponding first row of driving electrodes, and
the PCR chip further comprises a plurality of rows of second barrier members disposed along the first direction, each row of the second barrier members corresponds to a second row of driving electrodes, the second row of driving electrodes is divided into a plurality of segments by the second barrier members corresponding to the second row of driving electrodes, wherein a plurality of driving electrodes are disposed in each segment of driving electrodes, the second barrier members are disposed on a surface of the sealing substrate proximal to the PCR substrate, and a length direction of the second barrier members is a column direction.

15. The PCR chip of claim 12, wherein the sample inlet hole and the sample outlet hole are disposed in the sealing substrate and penetrate through the sealing substrate.

16. The PCR chip of claim 12, wherein the PCR chip further comprises an oil inlet hole and an oil outlet hole communicating with a region corresponding to the amplification region.

17. The PCR chip of claim 16, wherein the oil inlet hole and the oil outlet hole are disposed in the sealing substrate and penetrate through the sealing substrate.

18. A PCR system, comprising:
the PCR substrate of claim 1;
a temperature control structure configured to control temperatures at different positions of the predetermined track; and
a capture unit configured to capture an image of liquid droplets to analyze a number of specific bases.

19. A liquid droplets pull-out method using the PCR substrate of claim 1, comprising:
injecting a sample into the injection region;
stretching the sample from the injection region to the stretching region to form a strip sample; and
cutting off the strip sample to form liquid droplets.

20. The liquid droplets pull-out method of claim 19, wherein the driving structure further comprises: a plurality of gate lines and a plurality of data lines disposed on the first base;
a part of intersection points of the gate lines and the data lines are a plurality of effective intersection points, the plurality of driving electrodes are disposed at positions of the plurality of effective intersection points, a switching element is further disposed at a position of each of the plurality of effective intersection points, a first terminal and a second terminal of the switching element are respectively coupled to the data line and the driving electrode at the effective intersection point, and the gate line at the effective intersection point is coupled to a control electrode of the switching element;
the injecting the sample into the injection region comprises: providing a turn-on voltage to the gate lines corresponding to the driving electrodes in the injection region, providing an effective voltage to the data lines corresponding to the driving electrodes in the injection region, and providing an ineffective voltage to remaining data lines corresponding to the driving electrodes in the injection region;
the stretching the sample from the injection region to the stretching region to form the strip sample comprises: providing a turn-on voltage to the gate lines corresponding to the driving electrodes in the stretching region, and sequentially providing an effective voltage to corresponding data lines in the stretching region along a direction from the injection region to the stretching region; and
the cutting off the strip sample to form liquid droplets comprises: providing a turn-on voltage to the gate lines corresponding to the driving electrodes in the stretching region, providing an ineffective voltage to continuous data lines at the middle position in the stretching region, and sequentially providing an effective voltage to the data lines at two sides of the continuous data lines in the stretching region along the direction distal to the continuous data lines.
